(19) United States
(12) Patent Application Publication
(10) Pub. No.: US 2018/0168968 A1
(43) Pub. Date: Jun. 21, 2018

(54) AGENT AND METHOD FOR THE TEMPORARY RESHAPING OF KERATINOUS FIBERS

(71) Applicant: Henkel AG & Co. KGaA, Duesseldorf (DE)

(72) Inventors: Thorsten Knappe, Schenefeld (DE); Thea Kroehnke, Hamburg (DE)

(73) Assignee: Henkel AG & Co. KGaA, Duesseldorf (DE)

(21) Appl. No.: 15/841,302

(22) Filed: Dec. 14, 2017

(30) Foreign Application Priority Data
Dec. 21, 2016 (DE) 10 2016 225 873.7

(51) Int. Cl. A61K 8/34; A61K 8/92; A61K 8/04; A61Q 5/06

(52) U.S. Cl. CPC A61K 8/34; A61K 8/927; A61Q 5/06; A61K 8/342; A61K 8/046; A61K 8/922

(57) ABSTRACT

Cosmetic compositions or agents, the utilization thereof, and methods for the use of in temporary reshaping of keratinous fibers are provided. In an exemplary embodiment, a cosmetic composition includes from about 0.5 to about 20 wt. % wax, from about 1.0 to about 8.0 wt. % non-ionic emulsifier, from about 10 to about 25 wt. % ethanol, and from about 30 to about 80 wt. % propellant, all in relation to the overall weight of the cosmetic composition.

### CROSS-REFERENCE TO RELATED APPLICATION

This application claims priority to German Patent Application No. 10 2016 225 873.7, filed Dec. 21, 2016, which is incorporated herein by reference in its entirety.

### TECHNICAL FIELD

The application relates to the technical field of the temporary reshaping of keratinous fibers, in particular human hair.

### BACKGROUND

Styling agents for the reshaping of keratinous fibers have been known for some time, and are used in various embodiments for the design, enhancement and fixing of hairstyles that can only be obtained with many hair types through the use of solidifying active ingredients. Hair-treatment agents that serve to attain permanent or temporary shaping of the hair play a key role here.

Sprayable products as well as product types that can be worked into the hair by employing a comb or with the fingers are suitable for the temporary shaping of keratinous fibers. The latter product group comprises oils as well as waxes, gels, creams and powders with waxes or wax-type products enjoying growing popularity in some consumer segments.

Furthermore, cosmetic hair waxes have the advantages of the high level of hair hold attained with them, and the flexibility of the resulting hairstyles. One major disadvantage of hair waxes is their poor ability to be distributed on the hair, due to their viscosity.

Sprayable hair waxes provide a way of resolving this supposed dichotomy of effective hold and applicability. For instance, German patent applications DE 101 38 093 A1 and DE 10 2005 060 435 A1 describe cosmetic wax hairsprays based on a wax-emulsifier mixture.

An ethanol-based hairspray wax is described in German patent application DE 10 2010 055 842 A1.

The sprayable hair waxes known from the state of the art require further improvement in terms of their effective hold as well as their applicability and sprayability. This particularly applies with regard to the tendency of sprayable hair waxes to clog the spraying systems of common aerosol cans.

### BRIEF SUMMARY

Cosmetic compositions or agents and methods for the temporary reshaping of keratinous fibers are provided. In an exemplary embodiment, a cosmetic composition includes from about 0.5 to about 20 wt. % wax, from about 1.0 to about 8.0 wt. % non-ionic emulsifier; from about 10 to about 25 wt. % ethanol; and from about 30 to about 80 wt. % propellant, all in relation to the overall weight of the cosmetic composition.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The task of the present disclosure was therefore to provide hair-treatment agents in the form of sprayable wax preparations for temporary shaping that are exemplified by a good hairstyle hold, a high level of flexibility and the visible cosmetic effect that is typical of waxes. In addition, the wax preparations should not tend to clog the spraying systems of common aerosol cans.

It has been established that these tasks can be solved by employing a specific ethanol-based wax formulation.

The present disclosure provides:

1. A cosmetic composition comprising, in relation to its overall weight,
a) from about 0.5 to about 20 wt. % wax
b) from about 1.0 to about 8.0 wt. % non-ionic emulsifier
c) from about 10 to about 25 wt. % ethanol
d) from about 30 to about 80 wt. % propellant.
2. Cosmetic composition according to item 1, with the composition comprising from about 1.0 to about 15 wt. %, such as from about 3.0 to about 10 wt. % wax in relation to its overall weight.
3. Cosmetic composition according to one of the preceding items, with the composition comprising as wax at least one wax from the group of beeswax, carnauba wax, candelilla wax, and paraffin wax, such as from the group of beeswax, carnauba wax, and mixtures thereof.
4. Cosmetic composition according to one of the preceding items, with the composition comprising from about 1.5 to about 6.0 wt. %, such as from about 2.0 to about 5.0 wt. % non-ionic emulsifier in relation to its overall weight.
5. Cosmetic composition according to one of the preceding items, with the composition comprising as a non-ionic emulsifier a non-ionic emulsifier from the group of polyalkoxylated fatty alcohols.
6. Cosmetic composition according to one of the preceding items, with the composition comprising as a non-ionic emulsifier at least one emulsifier from the group of
i) Ethylene-oxide adducts to saturated or unsaturated linear fatty alcohols each with from about 2 to about 50 mol of ethylene oxide per mol of fatty alcohol, such as from about 2 to about 30 mol of ethylene oxide per mol of fatty alcohol
ii) Ethylene oxide-propylene oxide adducts to linear fatty alcohols with from about 8 to about 30 carbon atoms, for example the adducts of from about 2 to about 50 mol of ethylene oxide and from about 1 to about 5 mol propylene oxide to linear fatty alcohols with from about 12 to about 18 carbon atoms.
7. Cosmetic composition according to one of the preceding items, with the composition comprising from about 12 to about 22 wt. %, such as from about 14 to about 20 wt. % ethanol in relation to its overall weight.
8. Cosmetic composition according to one of the preceding items, with the composition comprising from about 40 to about 60 wt. %, such as from about 45 to about 55 wt. % propellant in relation to its overall weight.
9. Cosmetic composition according to one of the preceding items, with the composition comprising a propellant from the group of propane, propane/butane mixtures and dimethyl ether, for example dimethyl ether.
10. Cosmetic composition according to one of the preceding items, with the weight ratio of wax to ethanol being from about 1:1 to about 1:7, such as from about 1:1 to about 1:5.
11. Cosmetic composition according to one of the preceding items, with the weight ratio of wax to propellant being from about 1:3 to about 1:16, such as from about 1:5 to about 1:12.
12. Cosmetic composition according to one of the preceding items, with the composition furthermore comprising from about 0.5 to about 5.0 wt. %, such as from about 1.0 to about 4.0 wt. %, for example from about 1.5 to about 3.5 wt. % film-forming polymer.
13. Cosmetic composition according to item 11, with the film-forming polymer being selected from the group of non-ionic polymers.
14. Cosmetic composition, comprising
a) from about 3.0 to about 10 wt. % wax from the group of beeswax, carnauba wax, candelilla wax, montan wax and microcrystalline waxes
b) from about 2.0 to about 5.0 wt. % non-ionic emulsifier from the group of
i) Ethylene-oxide adducts to saturated or unsaturated linear fatty alcohols each with from about 2 to about 50 mol of ethylene oxide per mol of fatty alcohol, such as from about 2 to about 30 mol of ethylene oxide per mol of fatty alcohol
ii) Ethylene oxide-propylene oxide adducts to linear fatty alcohols with from about 8 to about 30 carbon atoms, for example the adducts of from about 2 to about 50 mol of ethylene oxide and from about 1 to about 5 mol of propylene oxide to linear fatty alcohols with from about 12 to about 18 carbon atoms
c) from about 14 to about 20 wt. % ethanol
d) from about 40 to about 60 wt. % dimethyl ether.
15. Cosmetic agent, comprising
a) a cosmetic composition according to one of items 1 to 14
b) an aerosol dispensing container.
16. Use of a cosmetic composition according to one of items 1 to 14 for the temporary reshaping of keratinous fibers.
17. Method for the temporary reshaping of keratinous fibers, for example human hair, in which the cosmetic composition according to one of items 1 to 14 is applied to keratinous fibers.

The compositions as contemplated herein exist in the form of an aerosol spray.

A first essential component of the compositions as contemplated herein is the wax. In relation to their total weight, exemplary cosmetic compositions contain from about 1.0 to about 15 wt. %, such as from about 3.0 to about 10 wt. % wax.

The exemplary wax has a melting point in the range of from about 40° C. to about 90° C., such as from about 50° C. to about 85° C., for example from about 50° C. to about 75° C.

In principle, it is possible to use all waxes that melt in the stated temperature range and are physiologically tolerable. Exemplary waxes can be allocated to the groups of natural waxes, mineral waxes and synthetic waxes. In exemplary embodiments, natural waxes are used. Exemplary individual waxes as contemplated herein are beeswax, carnauba wax, candelilla wax and paraffin wax, for example beeswax and carnauba wax as well as mixtures thereof.

The second essential component of the cosmetic compositions is the non-ionic emulsifier. Exemplary cosmetic compositions have a proportion of non-ionic emulsifier by weight of from about 1.5 to about 6.0 wt. %, such as from about 2.0 to about 5.0 wt. %.

With regard to the cosmetic properties and dosing properties of the compositions, non-ionic emulsifiers from the group of polyalkoxylated fatty alcohols may be particularly advantageous. Exemplary embodiments use non-ionic emulsifiers that are solid at room temperature (about 20° C.).

An exemplary group of emulsifiers is the polyalkoxylated fatty alcohols, with ethylene-oxide adducts to saturated or unsaturated linear fatty alcohols, each with from about 2 to about 50 mol of ethylene oxide per mol of fatty alcohol, such as from about 2 to about 30 mol of ethylene oxide per mol of fatty alcohol.

Exemplary emulsifiers are linear or branched, saturated or unsaturated C₁₂-C₃₀ alkanols, each of which are etherified with 2-4 ethylene-oxide units per molecule. Further exemplary emulsifiers are compounds with the INCI designations Steareth-2, Steareth-3, Steareth-4, Ceteth-2, Ceteth-3, Ceteth-4, Myristeth-2, Myristeth-3, Myristeth-4, Laureth-2, Laureth-3, Laureth-4, Trideceth-2, Trideceth-3 and Trideceth-4, as well as mixtures thereof.

A second group of exemplary emulsifiers is linear or branched, saturated or unsaturated C₁₂-C₃₀ alkanols, each of which are etherified with from about 20 to about 50 ethylene-oxide units per molecule. Examples of emulsifiers are compounds with the INCI designations Laureth-20, Laureth-25, Laureth-30, Laureth-40, Laureth-50, Myreth-20, Myreth-25, Myreth-30, Myreth-40, Myreth-50, Ceteth-20, Ceteth-25, Ceteth-30, Ceteth-40, Ceteth-50, Steareth-20, Steareth-21, Steareth-25, Steareth-30, Steareth-40, Steareth-50, Ceteareth-20, Ceteareth-25, Ceteareth-30, Ceteareth-40, Ceteareth-50, Oleth-20, Oleth-25, Oleth-30, Oleth-40 and Oleth-50.

A second group of polyalkoxylated fatty alcohols is the ethylene oxide-propylene oxide adducts to linear fatty alcohols with from about 8 to about 30 carbon atoms, such as the adducts of from about 2 to about 50 mol of ethylene oxide and from about 1 to about 5 mol of propylene oxide to linear fatty alcohols with from about 12 to about 18 carbon atoms. One example of this type of non-ionic emulsifier is the compound with the INCI designation PPG-5-Ceteth-20.

Examples of non-ionic emulsifier systems are compounds with the INCI designations Steareth-2, Steareth-21 and PPG-5-Ceteth-20 as well as mixtures of these emulsifiers.

The cosmetic compositions as contemplated herein contain ethanol as a further essential component. In an exemplary embodiment, the proportion of ethanol by weight of the total weight of the cosmetic composition is from about 12 to about 22 wt. %, such as from about 14 to about 20 wt. %.

The cosmetic properties, but in particular the application properties, of the cosmetic compositions can be further improved by selecting specific weight ratios of wax to ethanol in certain embodiments. Weight ratios of wax to ethanol of from about 1:1 to about 1:7, such as from about 1:1 to about 1:5, may be particularly technically advantageous.

A final essential component of the cosmetic compositions is the propellant. It may be particularly cosmetically and technically advantageous if the propellant has a proportion by weight of the total weight of the composition of from about 40 to about 60 wt. %, such as from about 45 to about 55 wt. %, in certain embodiments.

Exemplary propellants (propellant gases) are selected from propane, propene, n-butane, isobutane, isobutene, n-pentane, pentene, isopentane, isopentene, methane, ethane, dimethyl ether, nitrogen, air, oxygen, nitrous oxide, dichlorofluoromethane, chlorodifluoromethane, chlorofluoromethane, 1,1,2,2-tetrachloro-1-fluoroethane, 1,1,1,2-tetrachloro-2-fluoroethane, 1,2,2-trichloro-1,1-difluoroethane, 1,1,2-trichloro-1,2-difluoroethane, 1,1,1-trichloro-2,2-difluoroethane, 2,2-dichloro-1,1,1-trifluoroethane, 1,2-dichloro-1,1,2-trifluoroethane, 2-chloro-1,1,1,2-tetrafluoroethane, 1-chloro-1,1,2,2-tetrafluoroethane, 1,1,2-trichloro-2-fluoroethane, 1,2-dichloro-1,2-difluoroethane, 1,2-dichloro-1,1-difluoroethane, 1-chloro-1,2,2-trifluoroethane, 2-chloro-1,1,1-trifluoroethane, 1-chloro-1,1,2-trifluoroethane, 1,2-dichloro-1-fluoroethane, 1,1-dichloro-1-fluoroethane, 2-chloro-1,1-difluoroethane, 1-chloro-1,1-difluoroethane, 1-chloro-2-fluoroethane, 1-chloro-1-fluoroethane, 2-chloro-1,1-difluoroethene, 1,1,1,3-tetrafluoroethane, heptafluoro-n-propane, perfluoroethane, monochlorodifluoromethane, 1,1-difluoroethane, both individually and in combination.

Certain embodiments use propane, propane/butane mixtures or dimethyl ether. Further exemplary embodiments use dimethyl ethers, for example dimethyl ether as a sole propellant.

As already described in the case of the weight ratio of wax to ethanol, the weight ratio of wax to propellant has also proved to be relevant to the cosmetic and technical properties of the compositions as contemplated herein. In certain embodiments, weight ratios of wax to propellant are from about 1:3 to about 1:16, such as from about 1:5 to about 1:12.

In summary, exemplary cosmetic compositions include a) from about 3.0 to about 10 wt. % wax from the group of beeswax, carnauba wax, candelilla wax, montan wax and microcrystalline waxes;

b) from about 2.0 to about 5.0 wt. % non-ionic emulsifier from the group of:

i) Ethylene-oxide adducts to saturated or unsaturated linear fatty alcohols each with from about 2 to about 50 mol of ethylene oxide per mol of fatty alcohol, such as from about 2 to about 30 mol of ethylene oxide per mol of fatty alcohol; and

ii) Ethylene oxide-propylene oxide adducts to linear fatty alcohols with from about 8 to about 30 carbon atoms, such as the adducts of from about 2 to about 50 mol of ethylene oxide and from about 1 to about 5 mol of propylene oxide to linear fatty alcohols with from about 12 to about 18 carbon atoms;

c) from about 14 to about 20 wt. % ethanol; and
d) from about 40 to about 60 wt. % dimethyl ether.

In addition to the essential components described above, the cosmetic compositions can contain further optional active substances or excipients.

To further increase their cosmetic effect, in addition to the essential components, exemplary compositions contain an anionic or non-ionic polymer, such as a film-forming, non-ionic polymer. The proportion of these polymers by weight may be limited to quantities of from about 0.5 to about 5.0 wt-%, such as from about 1.0 to about 4.0 wt. %, for example from about 1.5 to about 3.5 wt. %.

Examples of non-ionic polymers of this kind are:

Vinyl-pyrrolidone/vinyl-ester copolymers, as sold under the trade name Luviskol (BASF), for example. Luviskol VA 64 and Luviskol VA 73, respectively vinyl-pyrrolidone/vinyl-acetate copolymers, are exemplary non-ionic polymers.

Cellulose ethers such as hydroxypropyl cellulose, hydroxyethyl cellulose and methyl hydroxypropyl cellulose, as sold under the trade names Culminal and Benecel (AQUALON), for example.

Polyvinyl pyrrolidones, as sold under the designation Luviskol (BASF), for example. Exemplary film-forming polymers used on account of their cosmetic effect are the polyvinyl pyrrolidones (INCI designation: PVP) and the vinyl-pyrrolidone/vinyl-acetate copolymers (INCI designation VP/VA copolymer). Further optional active substances or excipients may include additional nourishing agents.

The agent can contain at least one protein hydrolysate and/or one of its derivatives as a nourishing agent, for example. Protein hydrolysates are product mixtures that are obtained as a result of the acid-, base- or enzyme-catalyzed degradation of proteins. As contemplated herein, the term protein hydrolysates also covers total hydrolysates as well as individual amino acids and their derivatives as well as mixtures of different amino acids. The molecular weight of exemplary protein hydrolysates that are usable as contemplated herein is from about 75 Daltons, the molecular weight for glycine, to about 200,000 Daltons; the molecular weight is from about 75 to about 50,000 Daltons, such as from about 75 to about 20,000 Daltons.

Furthermore, the agent as contemplated herein can contain a vitamin, a provitamin, a vitamin precursor and/or one of their derivatives as a nourishing agent. As contemplated herein, exemplary vitamins, provitamins and vitamin precursors are those that are usually assigned to groups A, B, C, E, F and H.

Further nourishing agents are panthenol, caffeine, nicotinamide and sorbitol.

Furthermore, the agents as contemplated herein can contain at least one plant extract as a nourishing agent, but also monosaccharides or oligosaccharides and/or lipids.

The composition of some further exemplary cosmetic compositions can be found in the tables below (figures in wt. % in relation to the total weight of the cosmetic agent unless otherwise stated).

| | Formula 1 | Formula 2 | Formula 3 | Formula 4 | Formula 5 |
|---|---|---|---|---|---|
| Wax | 0.5 to 20 | 1.0 to 15 | 1.0 to 15 | 3.0 to 10 | 3.0 to 10 |
| Non-ionic | 1.0 to 8.0 | 1.0 to 8.0 | 1.5 to 6.0 | 1.5 to 6.0 | 2.0 to 5.0 |
| emulsifier | | | | | |
| Ethanol | 10 to 25 | 10 to 25 | 12 to 22 | 12 to 22 | 14 to 20 |
| Propellant | 30 to 80 | 40 to 60 | 40 to 60 | 40 to 60 | 45 to 55 |
| Optional | ad 100 | ad 100 | ad 100 | ad 100 | ad 100 |
| additives | | | | | |

| | Formula 6 | Formula 7 | Formula 8 | Formula 9 | Formula 10 |
|---|---|---|---|---|---|
| Beeswax, | 0.5 to 20 | 1.0 to 15 | 1.0 to 15 | 3.0 to 10 | 3.0 to 10 |
| carnauba wax | | | | | |
| Non-ionic | 1.0 to 8.0 | 1.0 to 8.0 | 1.5 to 6.0 | 1.5 to 6.0 | 2.0 to 5.0 |
| emulsifier | | | | | |
| Ethanol | 10 to 25 | 10 to 25 | 12 to 22 | 12 to 22 | 14 to 20 |
| Propellant | 30 to 80 | 40 to 60 | 40 to 60 | 40 to 60 | 45 to 55 |
| Optional | ad 100 | ad 100 | ad 100 | ad 100 | ad 100 |
| additives | | | | | |

| | Formula | Formula | Formula | Formula | Formula |
|---|---|---|---|---|---|
| | 11 | 12 | 13 | 14 | 15 |
| Wax | 0.5 to 20 | 1.0 to 15 | 1.0 to 15 | 3.0 to 10 | 3.0 to 10 |
| Polyalkoxylated | 1.0 to 8.0 | 1.0 to 8.0 | 1.5 to 6.0 | 1.5 to 6.0 | 2.0 to 5.0 |
| fatty alcohol | | | | | |
| Ethanol | 10 to 25 | 10 to 25 | 12 to 22 | 12 to 22 | 14 to 20 |
| Propellant | 30 to 80 | 40 to 60 | 40 to 60 | 40 to 60 | 45 to 55 |
| Optional | ad 100 | ad 100 | ad 100 | ad 100 | ad 100 |
| additives | | | | | |

| | Formula | | Formula | | Formula | | Formula | | Formula | |
|---|---|---|---|---|---|---|---|---|---|---|
| | 16 | | 17 | | 18 | | 19 | | 20 | |
| Wax | 0.5 to | 20 | 1.0 to | 15 | 1.0 to | 15 | 3.0 to | 10 | 3.0 to | 10 |
| Non-ionic | 1.0 to | 8.0 | 1.0 to | 8.0 | 1.5 to | 6.0 | 1.5 to | 6.0 | 2.0 to | 5.0 |
| emulsifier | | | | | | | | | | |
| Ethanol | 10 to | 25 | 10 to | 25 | 12 to | 22 | 12 to | 22 | 14 to | 20 |
| Dimethyl | 30 to | 80 | 40 to | 60 | 40 to | 60 | 40 to | 60 | 45 to | 55 |
| ether | | | | | | | | | | |

| | | | | | |
|---|---|---|---|---|---|
| Optional | ad 100 | ad 100 | ad 100 | ad 100 | ad 100 |
| additives | | | | | |

| | Formula | | Formula | | Formula | | Formula | | Formula | |
|---|---|---|---|---|---|---|---|---|---|---|
| | 21 | | 22 | | 23 | | 24 | | 25 | |
| Beeswax, carnauba wax | 0.5 to | 20 | 1.0 to | 15 | 1.0 to | 15 | 3.0 to | 10 | 3.0 to | 10 |
| Polyalkoxylated fatty alcohol | 1.0 to | 8.0 | 1.0 to | 8.0 | 1.5 to | 6.0 | 1.5 to | 6.0 | 2.0 to | 5.0 |
| Ethanol | 10 to | 25 | 10 to | 25 | 12 to | 22 | 12 to | 22 | 14 to | 20 |
| Propellant | 30 to | 80 | 40 to | 60 | 40 to | 60 | 40 to | 60 | 45 to | 55 |

| | | | | | |
|---|---|---|---|---|---|
| Optional additives | ad 100 | ad 100 | ad 100 | ad 100 | ad 100 |

| | Formula | | Formula | | Formula | | Formula | | Formula | |
|---|---|---|---|---|---|---|---|---|---|---|
| | 26 | | 27 | | 28 | | 29 | | 30 | |
| Beeswax, | 0.5 to | 20 | 1.0 to | 15 | 1.0 to | 15 | 3.0 to | 10 | 3.0 to | 10 |
| carnauba | | | | | | | | | | |
| wax | | | | | | | | | | |
| Non-ionic | 1.0 to | 8.0 | 1.0 to | 8.0 | 1.5 to | 6.0 | 1.5 to | 6.0 | 2.0 to | 5.0 |
| emulsifier | | | | | | | | | | |
| Ethanol | 10 to | 25 | 10 to | 25 | 12 to | 22 | 12 to | 22 | 14 to | 20 |
| Dimethyl | 30 to | 80 | 40 to | 60 | 40 to | 60 | 40 to | 60 | 45 to | 55 |
| ether | | | | | | | | | | |

| | | | | | |
|---|---|---|---|---|---|
| Optional | ad 100 | ad 100 | ad 100 | ad 100 | ad 100 |
| additives | | | | | |

| | Formula | | Formula | | Formula | | Formula | | Formula | |
|---|---|---|---|---|---|---|---|---|---|---|
| | 31 | | 32 | | 33 | | 34 | | 35 | |
| Beeswax, carnauba wax | 0.5 to | 20 | 1.0 to | 15 | 1.0 to | 15 | 3.0 to | 10 | 3.0 to | 10 |
| Polyalkoxylated fatty alcohol | 1.0 to | 8.0 | 1.0 to | 8.0 | 1.5 to | 6.0 | 1.5 to | 6.0 | 2.0 to | 5.0 |
| Ethanol | 10 to | 25 | 10 to | 25 | 12 to | 22 | 12 to | 22 | 14 to | 20 |
| Dimethyl ether | 30 to | 80 | 40 to | 60 | 40 to | 60 | 40 to | 60 | 45 to | 55 |

| | | | | | |
|---|---|---|---|---|---|
| Optional additives | ad 100 | ad 100 | ad 100 | ad 100 | ad 100 |

| | Formula | | Formula | | Formula | | Formula | | Formula | |
|---|---|---|---|---|---|---|---|---|---|---|
| | 36 | | 37 | | 38 | | 39 | | 40 | |
| Wax | 0.5 to | 20 | 1.0 to | 15 | 1.0 to | 15 | 3.0 to | 10 | 3.0 to | 10 |
| Non-ionic emulsifier | 1.0 to | 8.0 | 1.0 to | 8.0 | 1.5 to | 6.0 | 1.5 to | 6.0 | 2.0 to | 5.0 |
| Film-forming, non-ionic polymer | 0.5 to | 5.0 | 0.5 to | 5.0 | 1.0 to | 4.0 | 1.0 to | 4.0 | 1.5 to | 3.5 |
| Ethanol | 10 to | 25 | 10 to | 25 | 12 to | 22 | 12 to | 22 | 14 to | 20 |
| Propellant | 30 to | 80 | 40 to | 60 | 40 to | 60 | 40 to | 60 | 45 to | 55 |

| | | | | | |
|---|---|---|---|---|---|
| Optional additives | ad 100 | ad 100 | ad 100 | ad 100 | ad 100 |

| | Formula | | Formula | | Formula | | Formula | | Formula | |
|---|---|---|---|---|---|---|---|---|---|---|
| | 41 | | 42 | | 43 | | 44 | | 45 | |
| Beeswax, carnauba wax | 0.5 to | 20 | 1.0 to | 15 | 1.0 to | 15 | 3.0 to | 10 | 3.0 to | 10 |
| Non-ionic emulsifier | 1.0 to | 8.0 | 1.0 to | 8.0 | 1.5 to | 6.0 | 1.5 to | 6.0 | 2.0 to | 5.0 |
| Film-forming, non-ionic polymer | 0.5 to | 5.0 | 0.5 to | 5.0 | 1.0 to | 4.0 | 1.0 to | 4.0 | 1.5 to | 3.5 |
| Ethanol | 10 to | 25 | 10 to | 25 | 12 to | 22 | 12 to | 22 | 14 to | 20 |
| Propellant | 30 to | 80 | 40 to | 60 | 40 to | 60 | 40 to | 60 | 45 to | 55 |

| | | | | | |
|---|---|---|---|---|---|
| Optional additives | ad 100 | ad 100 | ad 100 | ad 100 | ad 100 |

| | Formula | | Formula | | Formula | | Formula | | Formula | |
|---|---|---|---|---|---|---|---|---|---|---|
| | 46 | | 47 | | 48 | | 49 | | 50 | |
| Wax | 0.5 to | 20 | 1.0 to | 15 | 1.0 to | 15 | 3.0 to | 10 | 3.0 to | 10 |
| Polyalkoxylated fatty | 1.0 to | 8.0 | 1.0 to | 8.0 | 1.5 to | 6.0 | 1.5 to | 6.0 | 2.0 to | 5.0 |
| alcohol | | | | | | | | | | |
| Film-forming, non-ionic polymer | 0.5 to | 5.0 | 0.5 to | 5.0 | 1.0 to | 4.0 | 1.0 to | 4.0 | 1.5 to | 3.5 |
| Ethanol | 10 to | 25 | 10 to | 25 | 12 to | 22 | 12 to | 22 | 14 to | 20 |
| Propellant | 30 to | 80 | 40 to | 60 | 40 to | 60 | 40 to | 60 | 45 to | 55 |

| | | | | | |
|---|---|---|---|---|---|
| Optional additives | ad 100 | ad 100 | ad 100 | ad 100 | ad 100 |

| | Formula | | Formula | | Formula | | Formula | | Formula | |
|---|---|---|---|---|---|---|---|---|---|---|
| | 51 | | 52 | | 53 | | 54 | | 55 | |
| Wax | 0.5 to | 20 | 1.0 to | 15 | 1.0 to | 15 | 3.0 to | 10 | 3.0 to | 10 |
| Non-ionic emulsifier | 1.0 to | 8.0 | 1.0 to | 8.0 | 1.5 to | 6.0 | 1.5 to | 6.0 | 2.0 to | 5.0 |
| Film-forming, non-ionic polymer | 0.5 to | 5.0 | 0.5 to | 5.0 | 1.0 to | 4.0 | 1.0 to | 4.0 | 1.5 to | 3.5 |
| Ethanol | 10 to | 25 | 10 to | 25 | 12 to | 22 | 12 to | 22 | 14 to | 20 |
| Dimethyl ether | 30 to | 80 | 40 to | 60 | 40 to | 60 | 40 to | 60 | 45 to | 55 |

| | | | | | |
|---|---|---|---|---|---|
| Optional additives | ad 100 | ad 100 | ad 100 | ad 100 | ad 100 |

| | Formula | | Formula | | Formula | | Formula | | Formula | |
|---|---|---|---|---|---|---|---|---|---|---|
| | 56 | | 57 | | 58 | | 59 | | 60 | |
| Beeswax, carnauba wax | 0.5 to | 20 | 1.0 to | 15 | 1.0 to | 15 | 3.0 to | 10 | 3.0 to | 10 |
| Polyalkoxylated fatty alcohol | 1.0 to | 8.0 | 1.0 to | 8.0 | 1.5 to | 6.0 | 1.5 to | 6.0 | 2.0 to | 5.0 |
| Film-forming, non-ionic polymer | 0.5 to | 5.0 | 0.5 to | 5.0 | 1.0 to | 4.0 | 1.0 to | 4.0 | 1.5 to | 3.5 |
| Ethanol | 10 to | 25 | 10 to | 25 | 12 to | 22 | 12 to | 22 | 14 to | 20 |
| Propellant | 30 to | 80 | 40 to | 60 | 40 to | 60 | 40 to | 60 | 45 to | 55 |

| | | | | | |
|---|---|---|---|---|---|
| Optional additives | ad 100 | ad 100 | ad 100 | ad 100 | ad 100 |

| | Formula | | Formula | | Formula | | Formula | | Formula | |
|---|---|---|---|---|---|---|---|---|---|---|
| | 61 | | 62 | | 63 | | 64 | | 65 | |
| Beeswax, carnauba wax | 0.5 to | 20 | 1.0 to | 15 | 1.0 to | 15 | 3.0 to | 10 | 3.0 to | 10 |
| Non-ionic emulsifier | 1.0 to | 8.0 | 1.0 to | 8.0 | 1.5 to | 6.0 | 1.5 to | 6.0 | 2.0 to | 5.0 |
| Film-forming, non-ionic polymer | 0.5 to | 5.0 | 0.5 to | 5.0 | 1.0 to | 4.0 | 1.0 to | 4.0 | 1.5 to | 3.5 |
| Ethanol | 10 to | 25 | 10 to | 25 | 12 to | 22 | 12 to | 22 | 14 to | 20 |
| Dimethyl ether | 30 to | 80 | 40 to | 60 | 40 to | 60 | 40 to | 60 | 45 to | 55 |

| | | | | | |
|---|---|---|---|---|---|
| Optional additives | ad 100 | ad 100 | ad 100 | ad 100 | ad 100 |

| | Formula | | Formula | | Formula | | Formula | | Formula | |
|---|---|---|---|---|---|---|---|---|---|---|
| | 66 | | 67 | | 68 | | 69 | | 70 | |
| Beeswax, carnauba wax | 0.5 to | 20 | 1.0 to | 15 | 1.0 to | 15 | 3.0 to | 10 | 3.0 to | 10 |
| Polyalkoxylated fatty alcohol | 1.0 to | 8.0 | 1.0 to | 8.0 | 1.5 to | 6.0 | 1.5 to | 6.0 | 2.0 to | 5.0 |
| Film-forming, non-ionic polymer | 0.5 to | 5.0 | 0.5 to | 5.0 | 1.0 to | 4.0 | 1.0 to | 4.0 | 1.5 to | 3.5 |
| Ethanol | 10 to | 25 | 10 to | 25 | 12 to | 22 | 12 to | 22 | 14 to | 20 |
| Dimethyl ether | 30 to | 80 | 40 to | 60 | 40 to | 60 | 40 to | 60 | 45 to | 55 |

| | | | | | |
|---|---|---|---|---|---|
| Optional additives | ad 100 | ad 100 | ad 100 | ad 100 | ad 100 |

| | Formula | | Formula | | Formula | | Formula | | Formula | |
|---|---|---|---|---|---|---|---|---|---|---|
| | 71 | | 72 | | 73 | | 74 | | 75 | |
| Beeswax, carnauba wax | 0.5 to | 20 | 1.0 to | 15 | 1.0 to | 15 | 3.0 to | 10 | 3.0 to | 10 |
| Polyalkoxylated fatty alcohol * | 1.0 to | 8.0 | 1.0 to | 8.0 | 1.5 to | 6.0 | 1.5 to | 6.0 | 2.0 to | 5.0 |
| Film-forming, non-ionic | 0.5 to | 5.0 | 0.5 to | 5.0 | 1.0 to | 4.0 | 1.0 to | 4.0 | 1.5 to | 3.5 |
| polymer | | | | | | | | | | |
| Ethanol | 10 to | 25 | 10 to | 25 | 12 to | 22 | 12 to | 22 | 14 to | 20 |
| Propellant | 30 to | 80 | 40 to | 60 | 40 to | 60 | 40 to | 60 | 45 to | 55 |

| | | | | | |
|---|---|---|---|---|---|
| Optional additives | ad 100 | ad 100 | ad 100 | ad 100 | ad 100 |

* From the group Steareth-2, Steareth-21 and PPG-5-Ceteth-20

| | Formula | | Formula | | Formula | | Formula | | Formula | |
|---|---|---|---|---|---|---|---|---|---|---|
| | 76 | | 77 | | 78 | | 79 | | 80 | |
| Beeswax, carnauba wax | 0.5 to | 20 | 1.0 to | 15 | 1.0 to | 15 | 3.0 to | 10 | 3.0 to | 10 |
| Polyalkoxylated fatty alcohol | 1.0 to | 8.0 | 1.0 to | 8.0 | 1.5 to | 6.0 | 1.5 to | 6.0 | 2.0 to | 5.0 |
| Film-forming, non-ionic | 0.5 to | 5.0 | 0.5 to | 5.0 | 1.0 to | 4.0 | 1.0 to | 4.0 | 1.5 to | 3.5 |
| polymer ** | | | | | | | | | | |
| Ethanol | 10 to | 25 | 10 to | 25 | 12 to | 22 | 12 to | 22 | 14 to | 20 |
| Propellant | 30 to | 80 | 40 to | 60 | 40 to | 60 | 40 to | 60 | 45 to | 55 |

| | | | | | |
|---|---|---|---|---|---|
| Optional additives | ad 100 | ad 100 | ad 100 | ad 100 | ad 100 |

** From the group of polyvinyl pyrrolidones (INCI designation: PVP) and of vinyl-pyrrolidone/vinyl-acetate copolymers (INCI designation VP/VA copolymer)

| | Formula | | Formula | | Formula | | Formula | | Formula | |
|---|---|---|---|---|---|---|---|---|---|---|
| | 81 | | 82 | | 83 | | 84 | | 85 | |
| Beeswax, carnauba wax | 0.5 to | 20 | 1.0 to | 15 | 1.0 to | 15 | 3.0 to | 10 | 3.0 to | 10 |
| Polyalkoxylated fatty alcohol * | 1.0 to | 8.0 | 1.0 to | 8.0 | 1.5 to | 6.0 | 1.5 to | 6.0 | 2.0 to | 5.0 |
| Film-forming, non-ionic | 0.5 to | 5.0 | 0.5 to | 5.0 | 1.0 to | 4.0 | 1.0 to | 4.0 | 1.5 to | 3.5 |
| polymer ** | | | | | | | | | | |
| Ethanol | 10 to | 25 | 10 to | 25 | 12 to | 22 | 12 to | 22 | 14 to | 20 |
| Propellant | 30 to | 80 | 40 to | 60 | 40 to | 60 | 40 to | 60 | 45 to | 55 |

| | | | | | |
|---|---|---|---|---|---|
| Optional additives | ad 100 | ad 100 | ad 100 | ad 100 | ad 100 |

* From the group Steareth-2, Steareth-21 and PPG-5-Ceteth-20
** From the group of polyvinyl pyrrolidones (INCI designation: PVP) and of vinyl-pyrrolidone/vinyl-acetate copolymers (INCI designation VP/VA copolymer)

| | Formula | | Formula | | Formula | | Formula | | Formula | |
|---|---|---|---|---|---|---|---|---|---|---|
| | 86 | | 87 | | 88 | | 89 | | 90 | |
| Beeswax, carnauba wax | 0.5 to | 20 | 1.0 to | 15 | 1.0 to | 15 | 3.0 to | 10 | 3.0 to | 10 |
| Polyalkoxylated fatty alcohol * | 1.0 to | 8.0 | 1.0 to | 8.0 | 1.5 to | 6.0 | 1.5 to | 6.0 | 2.0 to | 5.0 |
| Film-forming, non-ionic | 0.5 to | 5.0 | 0.5 to | 5.0 | 1.0 to | 4.0 | 1.0 to | 4.0 | 1.5 to | 3.5 |
| polymer ** | | | | | | | | | | |
| Ethanol | 10 to | 25 | 10 to | 25 | 12 to | 22 | 12 to | 22 | 14 to | 20 |
| Dimethyl ether | 30 to | 80 | 40 to | 60 | 40 to | 60 | 40 to | 60 | 45 to | 55 |

| | | | | | |
|---|---|---|---|---|---|
| Optional additives | ad 100 | ad 100 | ad 100 | ad 100 | ad 100 |

* From the group Steareth-2, Steareth-21 and PPG-5-Ceteth-20
** From the group of polyvinyl pyrrolidones (INCI designation: PVP) and of vinyl-pyrrolidone/vinyl-acetate copolymers (INCI designation VP/VA copolymer)

The cosmetic combination as contemplated herein may be combined with an aerosol dispensing container to form a cosmetic agent as contemplated herein.

Vessels made of metal (aluminum, tinplate, tin), protected or splinter-proof plastic or of glass that is coated on the outside with plastic are eligible as aerosol dispensing containers, with compression strength, tensile strength, corrosion resistance, ease of filling, aesthetic aspects, portability, printability etc. being factors in their selection. Special inner coatings ensure corrosion resistance in relation to the composition contained in the aerosol dispensing container.

Particularly good effects as contemplated herein are attained if the internal pressure of the aerosol pressure container is at least about 1.8 bar, in particular at least about 2.5 bar.

The aerosol dispensing container has a valve for dispensing of the aerosol. In an exemplary embodiment as contemplated herein, the valve has a valve head coated with a varnish or with a polymer plastic A, and a flexible element with resetting characteristics that resets the valve to the closing position (i.e., the resting position of the valve) after activation ceases. Corresponding exemplary cosmetic agents are those in which the aerosol dispensing container comprises a valve that has a valve cone and/or a flexible element with resetting characteristics that is/are coated with a varnish or a polymer plastic A.

In a further exemplary embodiment, the valve has a flexible element with resetting characteristics and/or a valve head made of at least one plastic B, such as an elastomer plastic. Here too, exemplary cosmetic agents are those in which the valve has a flexible element with resetting characteristics and/or a valve cone made of at least one plastic B, such as plastics B being elastomer plastics. Exemplary elastomer plastics are selected from butyl SH 74, nitrile B175 and nitrile BA 7402.

In a further exemplary embodiment as contemplated herein, the flexible element can be formed with resetting characteristics as a coil spring or compression spring.

In another exemplary embodiment as contemplated herein, the flexible element of the valve with resetting characteristics can be formed integrally with the valve cone and have flexible legs. This spring can be made of metal or plastic.

In an exemplary embodiment as contemplated herein, the valve cone and the flexible element are formed with resetting characteristics, such as the valve type Ariane M, available from Seaquist Perfect, in which the flexible element with resetting characteristics is formed integrally with the valve cone in the form of four elastic legs.

Exemplary valves have an inner-coated valve head, with the coating and the valve material being compatible with each other. If aluminum valves are used, their valve heads can be coated on the inside with Micoflex varnish, for example. If tin sheet valves are used, their valve heads can be coated on the inside with PET (polyethylene terephthalate), for example. The containers used, which can be made of tinplate or aluminum, for example, may also be varnished or coated on the inside in view of the possible corrosiveness of the cosmetic composition. An exemplary inner coating is an epoxy phenolic varnish, as available under the designation Hoba 7407 P, for instance.

In an exemplary embodiment, the valve is a valve of the type Aptar Arianne 0.32 mm. The spray nozzle WAZ from Aptar with the insert 6518 is used as the spray nozzle in certain embodiments.

As stated at the outset, the cosmetic compositions as contemplated herein are exemplified by particular advantages in terms of the temporary reshaping of keratinous fibers. A corresponding use of this cosmetic composition for temporary reshaping of keratinous fibers, especially human hair, is therefore a further object of this application.

A final object of this application is a method for the temporary reshaping of keratinous fibers, especially human hair, in which the cosmetic composition is applied to keratinous fibers.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic composition comprising:
a) from about 0.5 to about 20 wt. % wax;
b) from about 1.0 to about 8.0 wt. % non-ionic emulsifier;
c) from about 10 to about 25 wt. % ethanol; and
d) from about 30 to about 80 wt. % propellant, all in relation to the overall weight of the cosmetic composition.

2. The cosmetic composition according to claim 1, wherein the composition comprise from about 1.0 to about 15 wt. % wax in relation to the overall weight of the cosmetic composition.

3. The cosmetic composition according to claim 1, wherein the composition comprises as wax at least one wax from the group of beeswax, carnauba wax, candelilla wax, and paraffin wax, and mixtures thereof.

4. The cosmetic composition according to claim 1, wherein the composition comprises from about 1.5 to about 6.0 wt. % non-ionic emulsifier in relation to the overall weight of the cosmetic composition.

5. The cosmetic composition according to claim 1, wherein the composition comprises as a non-ionic emulsifier a non-ionic emulsifier from the group of polyalkoxylated fatty alcohols.

6. The cosmetic composition according to claim 1, wherein the composition comprises from about 12 to about 22 wt. % ethanol in relation to the overall weight of the cosmetic composition.

7. The cosmetic composition according to claim 1, wherein the composition comprises from about 40 to about 60 wt. % propellant in relation to the overall weight of the cosmetic composition.

8. A cosmetic agent comprising:
a) a cosmetic composition according to claim 1;
b) an aerosol dispensing container.

9. Use of a cosmetic composition according to claim 1 for the temporary reshaping of keratinous fibers.

10. Method for the temporary reshaping of keratinous fibers in which the cosmetic composition according to claim 1 is applied to the keratinous fibers.

11. The cosmetic composition according to claim 1, wherein the composition comprises from about 3.0 to about 10 wt. % wax in relation to the overall weight of the cosmetic composition.

12. The cosmetic composition according to claim 1, wherein the composition comprises from about 2.0 to about 5.0 wt. % non-ionic emulsifier in relation to the overall weight of the cosmetic composition.

13. The cosmetic composition according to claim 1, wherein the composition comprises from about 14 to about 20 wt. % ethanol in relation to the overall weight of the cosmetic composition.

14. The cosmetic composition according to claim 1, wherein the composition comprises from about 45 to about 55 wt. % propellant in relation to the overall weight of the cosmetic composition.
